(19) United States
(12) Patent Application Publication
(10) Pub. No.: US 2010/0087823 A1
(43) Pub. Date: Apr. 8, 2010

(54) PEDICLE PREPARATION DEVICE TO ASSIST IMPLANTATION OF PEDICLE SCREWS

(76) Inventor: Dimitriy G. Kondrashov, San Francisco, CA (US)

Correspondence Address: FLIESLER MEYER LLP, 650 CALIFORNIA STREET, 14TH FLOOR, SAN FRANCISCO, CA, 94108, US

(21) Appl. No.: 12/572,147

(22) Filed: Oct. 1, 2009

Related U.S. Application Data
(60) Provisional application No. 61/194,928, filed on Oct. 2, 2008.

(51) Int. Cl. A61B 17/00; A61F 5/00

(52) U.S. Cl. 606/79; 606/87

(57) ABSTRACT

A device to assist in preparing a pedicle of a patient to receive a pedicle screw comprises a shaft including a tip extending from a distal end of the shaft adapted to penetrate bone. An angle guide is connected with the shaft to provide an indication of shaft orientation along one or more planes relative to a pre-defined position.

### CLAIM TO PRIORITY

This application claims priority to U.S. Provisional Application No. 61/194,928, filed Oct. 2, 2008, entitled “ANGLE-SPECIFIC PEDICLE PROBE FOR INSERTION OF SPINE PEDICLE SCREWS,” incorporated herein by reference.

### TECHNICAL FIELD

Embodiments of the present invention relate to pedicle preparation devices to assist implantation of pedicle screws and methods of using such pedicle preparation devices.

### BACKGROUND OF INVENTION

Degenerative changes of the spinal column are the most common underlying cause of chronic back pain. The peak incidence of symptoms occurs between the ages of 30 and 50, a period of life during which the most strenuous job and sports-related activities occur. As individuals age, degenerative changes accumulate, affecting the vertebral discs, vertebra, facet joints, and ligaments in the thoracic and lumbar region and can lead to compression of spinal nerves and spinal nerve roots.

It can be desirable to reduce the compression of spinal nerves and spinal nerves by stabilizing the spinal column. The spinal column can be stabilized by isolating spinal motion segment and restricting relative motion between adjacent vertebrae. Alternatively, when conservative treatment fails to control the pain of degenerative disc disease, stenosis and spondylolisthesis, a common surgical approach is spinal fusion; over 200,000 spinal fusions are performed each year. The disc and other material that may be compressing nerve roots are removed and the vertebrae superior and inferior to the removed disc are fused. During the fusion process it is also desirable to restrict relative motion between vertebrae undergoing fusion while bone grows and fusion is complete.

Techniques to stabilize the spinal column and/or isolate vertebral motion segments commonly includes hardware attached to related vertebral bodies using pedicle screws. Pedicle screw implantation requires accurate alignment of the pedicle screw along the pedicle.

### BRIEF DESCRIPTION OF THE DRAWINGS

FIGS. 1A and 1B illustrate a pedicle probe in accordance with the prior art positioned to penetrate a pedicle.

FIG. 2 illustrates an embodiment of a pedicle preparation device in accordance with the present invention to assist in implantation of a pedicle screw.

FIG. 3 is a detailed view of a grip and head of an alternative embodiment of a pedicle preparation device in accordance with the present invention to assist in implantation of a pedicle screw.

FIG. 4 is a detailed view of a grip and head of a further embodiment of a pedicle preparation device in accordance with the present invention to assist in implantation of a pedicle screw.

FIG. 5 is a detailed view of a grip and head of a further embodiment of a pedicle preparation device in accordance with the present invention to assist in implantation of a pedicle screw.

FIG. 6A is a detailed view and FIG. 6B is a partial cross-sectional view of a tip and stop of an embodiment of a pedicle preparation device in accordance with the present invention to assist in implantation of a pedicle screw.

FIG. 7 is a detailed view of a tip and stop of an alternative embodiment of a pedicle preparation device in accordance with the present invention.

FIGS. 8A and 8B illustrate an embodiment of a pedicle preparation device in accordance with the present invention positioned to penetrate a pedicle.

FIGS. 8C and 8D illustrate the tip of the pedicle preparation device of FIGS. 8A and 8B penetrating the pedicle to a depth permitted by the stop.

FIG. 9 is a flowchart of a method to prepare a pedicle for a pedicle screw and implant the pedicle screw in accordance with an embodiment of the present invention.

FIG. 10 is a detailed view of features of a further embodiment of a pedicle preparation device in accordance with the present invention.

### DETAILED DESCRIPTION OF THE PREFERRED EMBODIMENTS

The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout. In addition, the first digit of a three digit reference number identifies the drawing in which the reference number first appears. Reference numerals used in a drawing may or may not be referenced in the detailed description specific to such drawing if the associated element is described elsewhere. Further, the terms “vertical” and “horizontal” are used throughout the detailed description to describe general orientation of structures relative to the spine of a human patient that is standing.

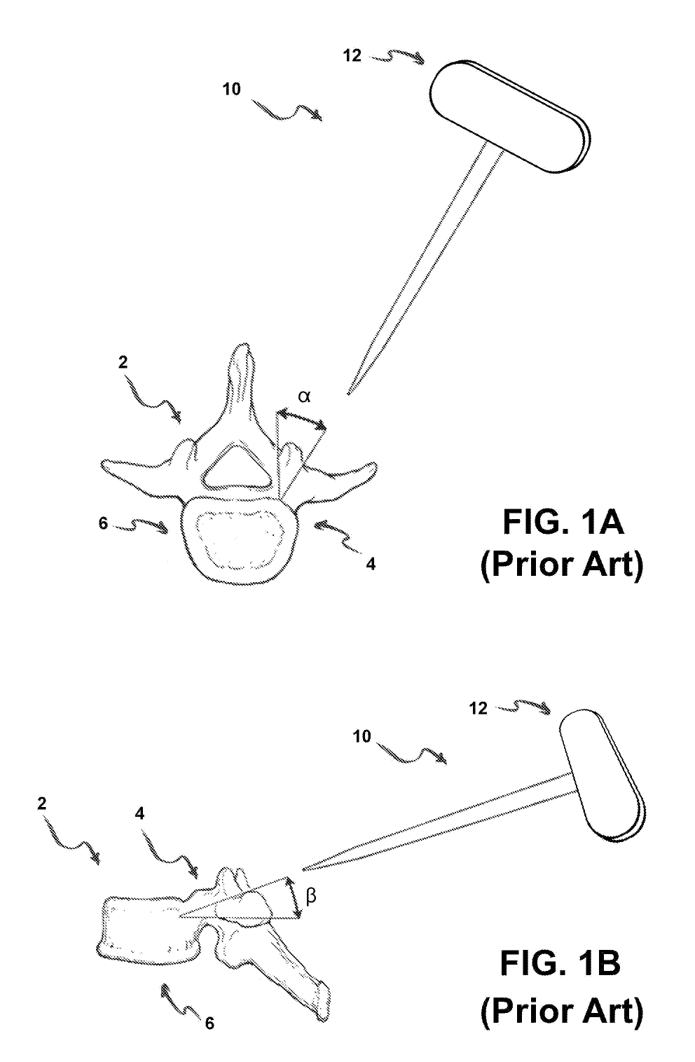

FIGS. 1A and 1B illustrate a pedicle probe 10 in accordance with the prior art positioned to penetrate a pedicle 4 of a vertebra in the thoracic region of the spinal column. The width of the pedicle 4 in the axial plane (the plane of the page in FIG. 1A) is narrower, and therefore allows less clearance space, than the height of the pedicle in the sagittal plane (the plane of the page in FIG. 1B) except in the lower lumbar spine. Pedicle size and angulation vary along the spinal column. For example, pedicles in the thoracolumbar region of the spinal column for an adult male can have a pedicle width that varies from approximately 5 mm at the T5 vertebral level to approximately 16 mm at the L5 vertebral level. Further, the axial pedicle angle α decreases descending caudally along the spinal column to the lumbar region. The axial pedicle angle α then increases as the lumbar spine is descended. The sagittal pedicle angle β is steep throughout the midthoracic spine and in the upper lumbar spine.

Multiple different techniques for implanting techniques have been practiced and/or proposed. Two of the more common techniques are computer-assisted pedicle screw placement, for example using fluoroscopic methods, and free-hand pedicle screw placement in which anatomic landmarks and specific entry sites are used to guide the surgeon. Commercially available systems for computer-assisted pedicle screw placement can be prohibitively expensive and/or unavailable. It is therefore desirable to improve the accuracy of free-hand pedicle screw placement.

Free-hand pedicle screw placement is performed using at least some of the following steps. First, an entry site to the target pedicle 4 is decorticated using a burr and a high-speed drill or a rongeur. A burr or awl is then used to penetrate the dorsal cortex of the pedicle 4. The pedicle probe 10, having a curved or straight tip, is then used to develop a path for a pedicle screw through the cancellous bone of the pedicle 4 and into the vertebral body 6. To develop the path, the surgeon typically grips the handle 12 of the pedicle probe 10 and the shaft near the pedicle 4 and advances the shaft into the pedicle 4. Advancement of the pedicle probe 10 is preferably smoothly and consistently directed at the correct axial pedicle angle α and sagittal pedicle angle β. The angles of insertion can be obtained from anatomical charts or alternatively calculated from pre-operative radiographs (e.g., from magnetic resonance imaging (MRI)), and/or a computed tomography (CT) scan. After the pedicle 4 has been probed and the trajectory and entry site are confirmed, a pedicle screw is implanted within the pedicle screw path (which can either be pre-tapped, or tapped while advancing the pedicle screw along the pedicle screw path).

The pedicle 4 includes a strong shell of cortical bone and a core of cancellous bone. An increase in resistance indicates abutment against cortical bone of the pedicle 4 or the thin covering of cortical bone surrounding the vertebral body 6. Sudden plunging (i.e., unexpected and/or uncontrolled progression of the tip through the bone) suggests breaking out of the pedicle 4 laterally which can occur if the pedicle probe 10 orientation deviates from the pedicle angles α, β. Plunging in a lateral direction can damage soft tissue or nerves. Plunging in a medial direction can damage the spinal cord. Embodiments of pedicle preparation devices in accordance with the present invention to assist in implantation of a pedicle screw can improve a surgeon's ability to accurately determine trajectory of a pedicle screw path, thereby reducing a risk of plunging that can damage soft tissue, nerves, or the spinal cord.

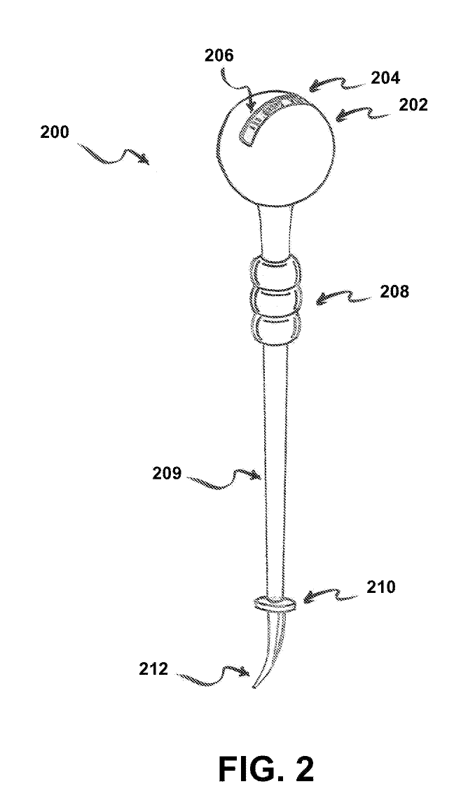

Refer to FIG. 2, an embodiment of a pedicle preparation device 200 in accordance with the present invention is shown comprising a shaft 209 including a tip 212 extending from a distal end of the shaft 209 adapted to penetrate bone and an angle guide 204 providing an indication of shaft orientation along a plane relative to a pre-defined position. The angle guide 204 as shown is including in a head 202 extending from the proximal end of the shaft 209. The head 202 is spherically shaped to be gripped in a surgeon's hand, allowing the surgeon, if desired, to apply pressure using the palm to urge the pedicle preparation device 200 along a trajectory aligned with the shaft 209. In an embodiment, the pre-defined position relative to which the shaft orientation is measurable lies along a line defined by the sagittal plane (as described above), the axial plane (also as described above), and/or the coronal plane. Preferably, the pre-defined position is designated the 0° position and the range of angles measurable by the angle guide at least extends the range of pedicle angulation of the vertebral levels to be probed (e.g., between 0° and 40° for the thoracolumbar region of the spinal column). The angle guide 204 can be calibrated to indicate the common angles encountered during the insertion of pedicle screws into spine, allowing the surgeon to know precisely the angle at which the pedicle probe is being inserted.

The pedicle preparation device 200 further comprises a stop 210 and a grip 208 arranged along the shaft 209 to assist guiding the trajectory of the shaft 209 and/or urging the tip 212 against the pedicle. The grip 208 is shown arranged along the shaft 209 closer to the head 202 than the tip 212; however, in other embodiments, the grip can be positioned closer along the shaft 209 to the tip 212 than the head 202. The grip 208 provides a contoured surface that can include grooves for improved handling of the pedicle preparation device 200 when compared with gripping the smooth, rounded shaft 209. The stop 210 restricts a depth to which the tip 212 can be urged into the pedicle. The stop 210 has a diameter sufficiently larger than the shaft 209 to obstruct movement within the pedicle screw path formed by the tip 212, and is sufficiently rigid to resist collapse from force applied along the shaft 209. The stop 210 can be positioned to allow a sufficient penetration depth for the tip 212 such that a satisfactory pedicle screw path can be formed while limiting potential damage during an instance of plunging that can result from an inaccurate trajectory. The stop 210 can be made transparent for the ease of visualization.

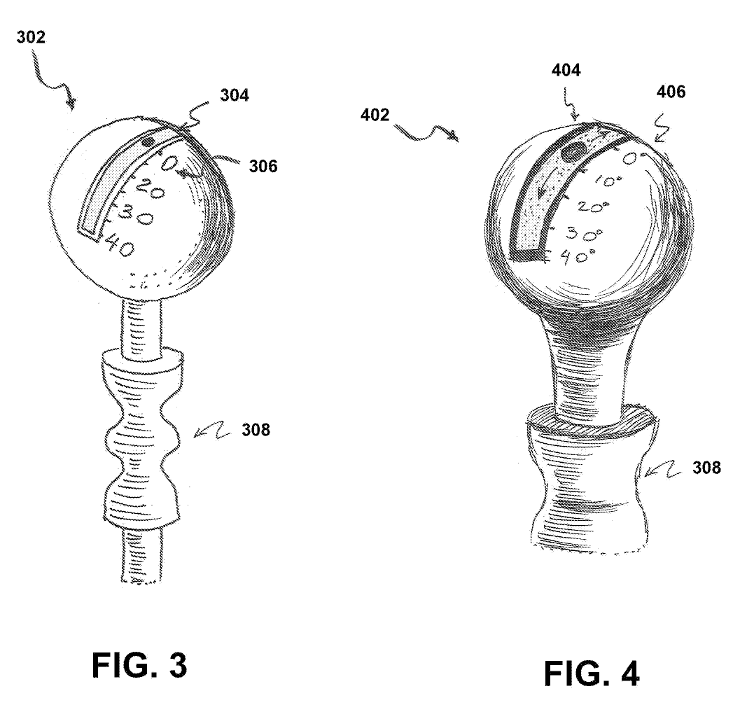

FIG. 3 illustrates a head 302 and grip 308 of an alternative embodiment of a pedicle preparation device in accordance with the present invention. The head 302 includes an angle guide 304 stylistically different from the angle guide 204 of FIG. 2, but both angle guides 204, 304 can be implemented as a spirit level comprising a gas bubble trapped in a viscous fluid (e.g., oil) or water held within a transparent vessel that prevents the gas bubble from moving outside the narrow bounds of the width of the gauge 206, 306. The gas bubble is positioned adjacent to a hash on the gauge 306, or behind a marking of a gauge 206 scribed on the transparent surface of the spirit level, indicating 0° when the pedicle preparation device 200, 300 is arranged in the pre-defined position. As the pedicle preparation device 200, 300 is tilted to orient the shaft at an angle divergent from that in the zero position, the bubble moves within the viscous fluid to indicate the angle by way of the gauge 206, 306. The grip 308 of the pedicle preparation device 300 of FIG. 3 is contoured to approximate the shape of a surgeon's fingers.

Referring to FIG. 4, a head 402 of a still further embodiment of a pedicle preparation device in accordance with the present invention is shown. The head 402 includes an angle guide 404 with a window and gauge 406 inscribed in a shell rotatable relative to the shaft to permit the gauge to be adjusted relative to the bubble to accommodate a change in the predefined position. As will be appreciated, spinal misalignment and malformation can result in vertebral bodies that are not properly aligned when the patient is in a prostrate position. An adjustable gauge can allow a surgeon to compensate for changes in the orientation of the vertebral body related to such misalignment so that the pedicle angles can be determined relative to the changed orientation (i.e., the angle is based on the anatomy of the target vertebra rather than the anatomical planes of the body).

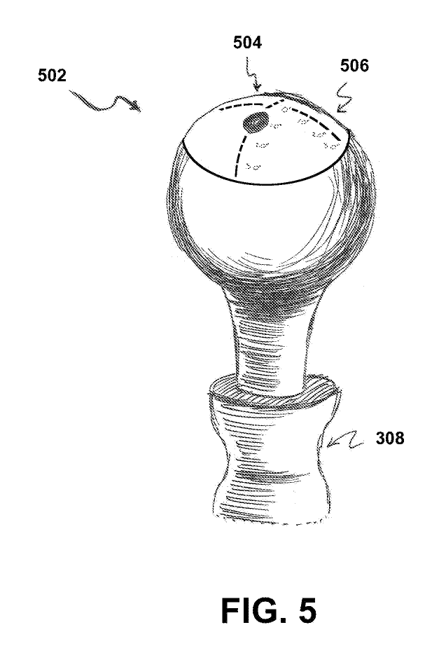

FIG. 5 illustrates a head 502 of a further embodiment of a pedicle preparation device in accordance with the present invention. The head 502 includes an angle guide 504 that permits measuring both the axial pedicle angle α and the sagittal pedicle angle β simultaneously. As shown, the angle guide 504 is implemented as a bulls-eye level, which is a type of spirit level comprising a gas bubble trapped in a viscous fluid held within a transparent vessel and allowing the gas bubble to move freely in the two dimensions. A gauge 506 is inscribed or otherwise formed on the head 502 to allow the pedicle angles to be measured.

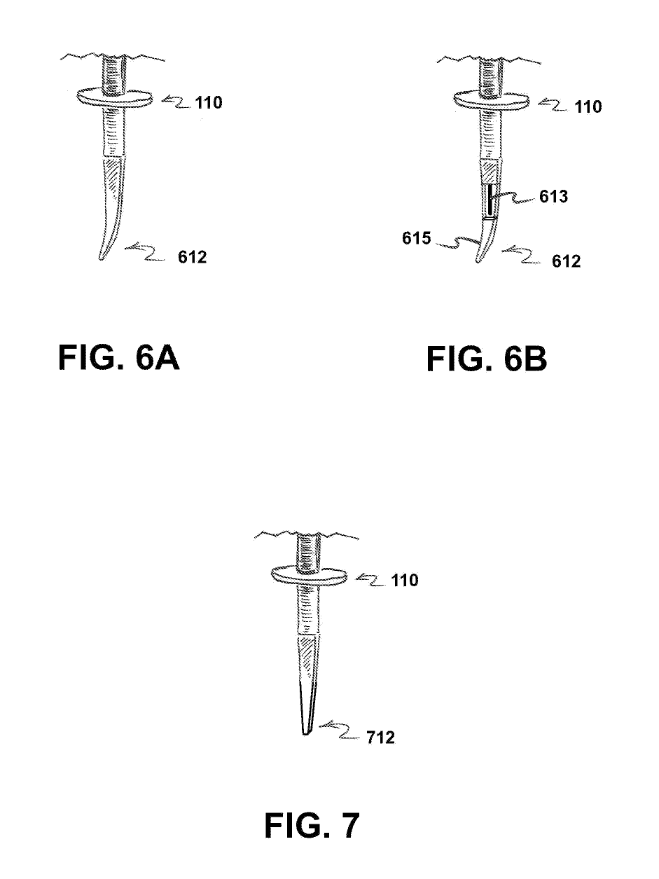

FIGS. 6A and 6B illustrate a tip 612 and stop 110 of an embodiment of a pedicle preparation device in accordance with the present invention. The tip 612 is curved and includes a relatively rigid core 613 with a less dense and comparatively more pliable outer structure 615 intended to yield slightly when coming into contact with cortical bone, thereby reducing a risk of unintentionally penetrating the cortical bone and plunging. In some embodiments, the pedicle preparation device is a one-time use device. In other embodiments, the outer structure 615 of the tip can be a one-time use structure while the rest of the pedicle preparation device is re-usable. In still further embodiments, the shaft can be removably connected with a head (e.g., by complementary screw threads). The shaft can be a one-time use structure while the head is can be removably connected with a new shaft for each use. FIG. 7 illustrates a tip 712 and stop 110 of an alternative embodiment of a pedicle preparation device in accordance with the present invention. The tip 712 is straight and aligned with the shaft. As shown, the stop 110 in both embodiments is a circular disk-like structure. In other embodiments, the stop can have some other shape. For example, the stop can be larger and have a shape that complements a structure of the vertebral body so that the pedicle preparation device must be aligned to approximately the correct orientation, or provided with a rough approximation.

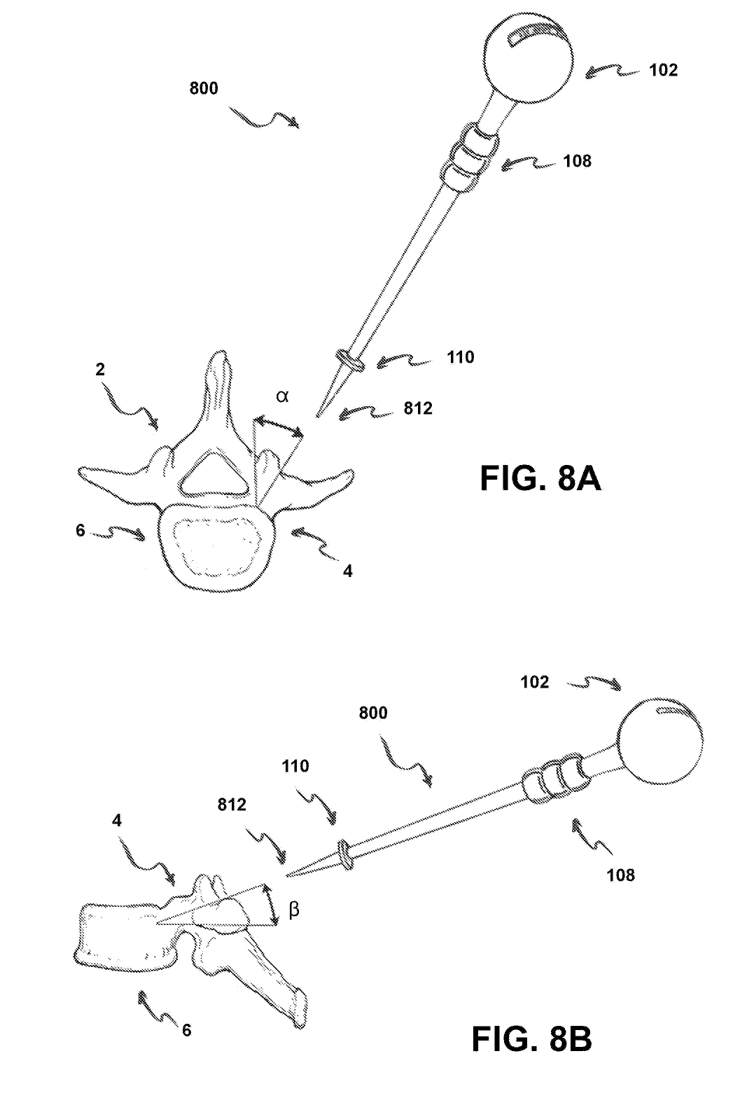

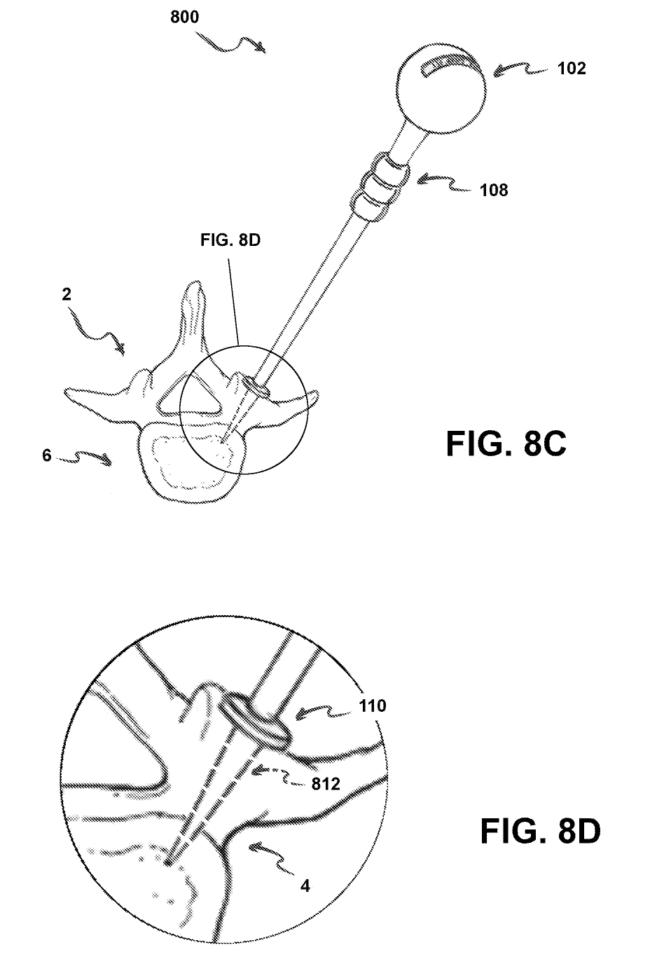

FIGS. 8A and 8B illustrate an embodiment of a pedicle preparation device 800 in accordance with the present invention positioned to penetrate a pedicle 4 of a vertebra in the thoracic region of the spinal column. The pedicle preparation device 800 comprises a head 102 including an angle guide 104 permitting measurement of angle along a single axis. After the vertebra 2 has been surgically prepared, the pedicle preparation device 800 is positioned by rotating the preparation device 800 so that the axis of the angle guide is parallel to the axial plane and perpendicular to the spinous process of the vertebra 2. The pedicle preparation device 800 is then tilted within the axial plane until the angle guide 104 measures the proper axial pedicle angle α. The pedicle preparation device 800 is then rotated so that the axis of the angle guide is parallel to the sagittal plane and parallel to the spinous process of the vertebra. The pedicle preparation device 800 is then tilted within the sagittal plane until the angle guide 104 measures the proper sagittal pedicle angle β.

FIGS. 8C and 8D illustrate the pedicle preparation device 800 of FIGS. 8A and 8B correctly oriented and seated within the pedicle, forming a pedicle screw hole. As can be seen particularly in FIG. 8D, a stop 110 prevents plunging and further prevents the tip 812 from penetrating too far into the pedicle 4. In an embodiment, the stop can be positioned at 45 or 50 mm from the tip for a device for use on an adult treatment or at 40 or 45 mm for a device for use in pediatric treatment. Those values represent common insertion depths in common practice. The stop improves the safety of pedicle screw insertion by providing a fool-proof dead stop which will avoid inadvertent plunging and a resultant injury to the vital neurovascular and visceral structures. Such injuries are extremely rare, but when they occur they can be catastrophic. The provides a safeguard to minimize injuries.

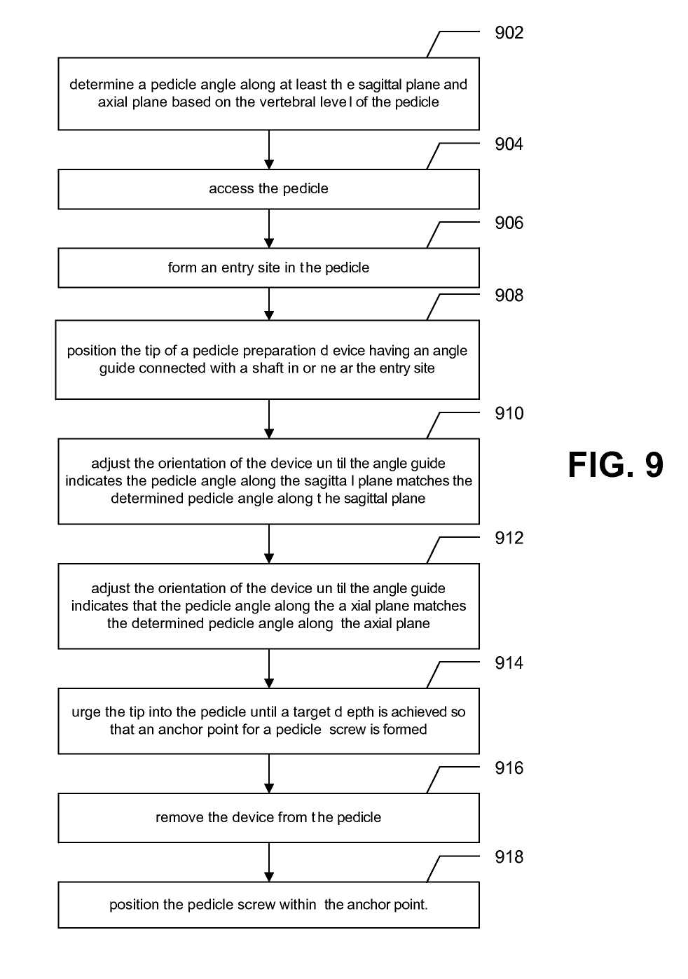

FIG. 9 is a flow chart of an embodiment of a method to assist in preparing a pedicle to receive a pedicle screw in accordance with the present invention. The method comprises using a pedicle preparation device, such as described above in FIGS. 2-8D, including a shaft having a tip extending from a distal end of the shaft to penetrate bone and an angle guide connected with the shaft to provide an indication of shaft orientation along one or more planes relative to a pre-defined position. A surgeon determines a pedicle angle along at least the sagittal plane and axial plane based on the vertebral level of the pedicle (Step 902). The surgeon can consult a reference manual, or alternatively can determine pedicle angles based on radiographs and/or a computed tomography (CT) scan. Once the surgeon has prepared, the target pedicle can be accessed (Step 904). An entry site to the pedicle is then formed in the pedicle for receiving the tip of the pedicle preparation device (Step 906). The entry site can be decorticated using a burr and a high-speed drill or a rongeur. A burr or awl can then be used to penetrate the dorsal cortex of the pedicle 4. The tip can then be positioned in or near the entry site (Step 908) and the orientation of the pedicle preparation device can be adjusted until the angle guide indicates the pedicle angle along the sagittal plane matches the determined pedicle angle along the sagittal plane (Step 910) and the pedicle angle along the axial plane matches the determined pedicle angle along the axial plane (Step 912).

Once the pedicle preparation device is properly oriented, the tip can then be urged into the pedicle until a target depth is achieved so that an anchor point for a pedicle screw is formed (Step 914). The target depth may be determined based on markings along the shaft, real-time imaging, a stop that limits the depth to which the tip can be urged, or any other technique known in the art. The pedicle preparation device is then removed (Step 916). After the trajectory and entry site are confirmed, a pedicle screw is implanted within the pedicle screw path (which can either be pre-tapped, or tapped while advancing the pedicle screw along the pedicle screw path) (Step 918).

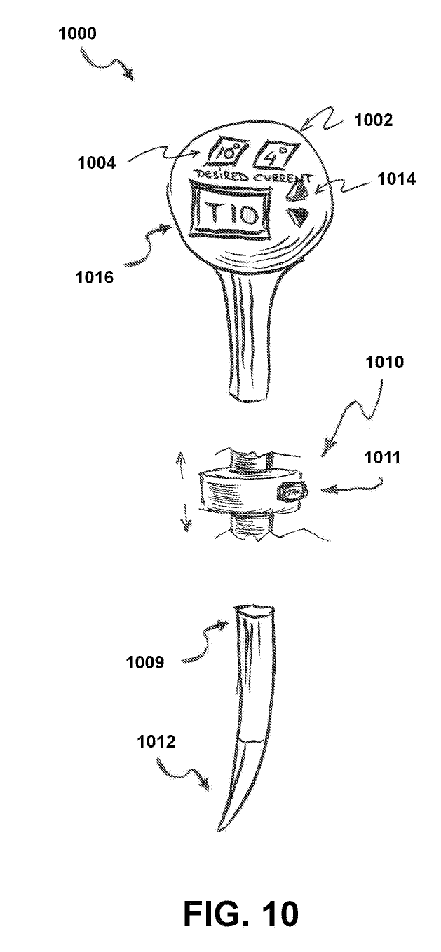

While pedicle preparations devices have been described above as using spirit levels as angle guides, embodiments of pedicle preparation devices in accordance with the present invention need not be limited to angle guides comprising spirit levels. For example, in some embodiments a digital inclinometer can be used to determine orientation of the pedicle preparation device shaft. Referring to FIG. 10, an embodiment of a pedicle preparation device in accordance with the present invention is shown comprising a digital inclinometer that uses an accelerometer or other electronics-based sensor for determining orientation. The head 1002 of the pedicle preparation device 1000 includes selection buttons 1014 and a display 1016 for identifying a target vertebral level. General reference values can be programmed into the pedicle preparation device 1000, or alternatively information from a radiograph and/or CT scan can be programmed into the pedicle preparation device 1000. As shown, the selection buttons 1014 allow the surgeon to advance up or down in vertebral level (for example, from T10 to T9 or T11). In other embodiments, some other input technique can be used to select the target vertebral body, such as a wireless keyboard communicatively coupled with the head or a reduced keypad (e.g., with C, T, L and 0-9) provided on the head. Additional displays 1004 illustrate a target orientation and a current orientation, and/or a delta value. The head 1002 can include fewer or more displays to communicate different information to a user and/or one or more transducer to communicate audible warnings or other information. As shown, a single pedicle angle is identified, but in other embodiments additional pedicle angles (i.e., in the sagittal, axial, and/or coronal planes) can be identified so that the pedicle preparation device 1000 need not be rotated when preparing the pedicle preparation device 1000 for insertion of the tip 1012 into an entry site. Potentially, a single pedicle finder may have one, two or three inclinometers to provide better 3-dimensional orientation during the pedicle screw insertion. To allow for vertebral rotation, the inclinometer can have an option to zero at any particular angle, for instance the angle of a given spinous process. This would make screw placement in the deformity cases easier.

The embodiment of FIG. 10 further includes a movable stop 1010. The stop 1010 can be repositioned along the shaft 1009 by depressing a button 1011 that causes the stop 1010 to disengage from the shaft 1009. The movable stop 1010 can allow the pedicle preparation device 1000 to be used at multiple vertebral levels with greater overall accuracy. In an embodiment, the adjustable stop can be position from 30 to 55 mm from the tip in 5 mm increments.

A pedicle preparation device with an angle guide can improve free-hand techniques for insertion of pedicle screws into at any vertebral level of the spinal column: the cervical, thoracic and lumbar areas. Further, a pedicle preparation device with an angle guide can reduce the need for computer navigation or fluoroscopy for those surgeons who do not utilize the free-hand technique. Depending on the surgeon's preference a handle can incorporate an angle guide that allows for measurement along one, two or three dimensions providing guidance in orthogonal planes.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalents.

1. A device to assist in preparing a pedicle of a patient to receive a pedicle screw comprising:
a shaft including a tip extending from a distal end of the shaft adapted to penetrate bone; and
an angle guide connected with the shaft, the angle guide providing an indication of shaft orientation along one or more planes relative to a pre-defined position.

2. The device of claim 1, further comprising:
a handle extending from a proximal end of the shaft, and
wherein the handle includes the angle guide.

3. The device of claim 1, further comprising:
a stop connected with the shaft to limit a depth to which the shaft is permitted to penetrate the pedicle when urged against the pedicle.

4. The device of claim 2, further comprising:
a grip connected with the shaft and arranged between the handle and the tip, the grip being shaped to generally receive one or more fingers of a user.

5. The device of claim 1, wherein the angle guide provides an indication of shaft orientation in the axial plane and the sagittal plane simultaneously.

6. The device of claim 1, wherein the angle guide includes one or both of an adjustable pre-defined position and an adjustable gauge.

7. The device of claim 1, wherein the angle guide is a spirit level.

8. The device of claim 1, wherein the angle guide is a bull's eye level.

9. The device of claim 1, wherein:
the angle guide includes an accelerometer to determine orientation and one or both of a visual and aural device to communicate orientation to a user.

12. The device of claim 1, wherein the tip is one of straight and curved.

13. The device of claim 1, wherein the tip is detachable from the shaft.

14. The device of claim 3, wherein a position of the stop along the shaft is adjustable.

15. The device of claim 9, further comprising one or more processors allowing a user to select a vertebral level and indicating a target orientation and an orientation determined based on the accelerometer.

16. The device of claim 12, wherein the tip includes a core and a shell, the shell being softer than the core.

17. A device to assist in preparing a pedicle of a patient to receive a pedicle screw comprising:
a shaft including a tip extending from a distal end of the shaft and adapted to penetrate bone and a handle connected with a proximal end of the shaft;
an angle guide associated with the handle to provide an indication of shaft orientation along one or more planes relative to a pre-defined position;
a stop connected with the shaft to limit a depth to which the shaft is permitted to penetrate the pedicle when urged against the pedicle; and
a grip connected with the shaft and arranged between the handle and the tip, the grip being shaped to generally receive one or more fingers of a user.

18. A system to assist in preparing a pedicle of a patient with a misaligned spine to receive a pedicle screw comprising:
a device including:
a shaft having a tip extending from a distal end of the shaft and adapted to penetrate bone, and
an angle guide connected with the shaft to provide an indication of shaft orientation along one or more planes relative to a pre-defined position; and
a jig adapted to be mated with a target vertebra of the misaligned spine;
wherein when the jig receives the device the pre-defined position is adjustable to allow the device to be used with standard reference charts of pedicle angles.

19. A method to assist in preparing a pedicle to receive a pedicle screw comprising:
using a device including a shaft having a tip extending from a distal end of the shaft to penetrate bone and an angle guide connected with the shaft to provide an indication of shaft orientation along one or more planes relative to a pre-defined position;
determining a pedicle angle along at least the sagittal plane and axial plane based on the vertebral level of the pedicle;
accessing the pedicle;
forming an entry site in the pedicle for receiving the tip;
positioning the tip in or near the entry site;
adjusting the orientation of the device until the angle guide indicates the pedicle angle along the sagittal plane matches the determined pedicle angle along the sagittal plane;
adjusting the orientation of the device until the angle guide indicates that the pedicle angle along the axial plane matches the determined pedicle angle along the axial plane;
urging the tip into the pedicle until a target depth is achieved so that an anchor point for a pedicle screw is formed;
removing the device from the pedicle; and
positioning the pedicle screw within the anchor point.

20. The method of claim 19, wherein the device includes a stop connected with the shaft to limit a depth to which the shaft is permitted to penetrate the pedicle when urged against the pedicle; and the method further comprising:
urging the tip into the pedicle until the stop contacts the pedicle.

21. The method of claim 19, determining a pedicle angle along at least the sagittal plane and axial plane based on the vertebral level of the pedicle includes imaging the patient's anatomy.
